(19) United States
(12) Patent Application Publication
(10) Pub. No.: US 2026/0207820 A1
(43) Pub. Date: Jul. 23, 2026

(54) INJECTABLE BONE IMPLANT COMPOSITIONS, KITS, AND RELATED METHODS

(71) Applicant: VIVORTE, INC., Louisville, KY (US)

(72) Inventor: Alyssa Osbourne, Louisville, KY (US)

(21) Appl. No.: 19/141,490

(22) PCT Filed: Dec. 21, 2023
(86) PCT No.: PCT/US2023/085334

§ 371 Date: Jun. 20, 2025

Related U.S. Application Data
(60) Provisional application No. 63/476,657, filed on Dec. 22, 2022.

(51) Int. Cl. A61L 27/36; A61F 2/28; A61F 2/30; A61F 2/46; A61L 27/02; A61L 27/12; A61L 27/20

(52) U.S. Cl. CPC A61L 27/3608; A61F 2/4601; A61L 27/025; A61L 27/12; A61L 27/20; A61L 27/3616; A61L 27/3695; A61F 2002/2835; A61F 2002/30062; A61F 2002/4645; A61F 2310/00293; A61F 2310/00353; A61F 2310/00359; A61L 2430/02

(57) ABSTRACT

A bone implant composition is provided that includes a viscous binder and a plurality of bone microparticles having a size that improves injectability of the bone implant compositions. Each of the bone microparticles can have a particle size of less than about 250 μm. The bone microparticles and viscous binder can further be combined with a biologically-resorbable cement and/or a hydration liquid. Kits including the bone implant compositions are further provided as well as methods of using the bone implant compositions to treat a bone defect in a subject. Methods of making the bone implant compositions include obtaining an amount of intact bone, milling the intact bone to produce a plurality of bone microparticles, and demineralizing the plurality of bone microparticles under defined conditions.

### RELATED APPLICATIONS

This application claims priority from U.S. Provisional Application Ser. No. 63/476,657, filed Dec. 22, 2022, the entire disclosure of which is incorporated herein by this reference.

### TECHNICAL FIELD

The presently-disclosed subject matter generally relates to injectable bone implant compositions, kits, and methods for making and using the bone implant compositions. In particular, certain embodiments of the presently-disclosed subject matter relate to injectable bone implant compositions and related kits and methods that make use of an injectable bone graft comprising a plurality of bone microparticles, a viscous binder, and/or a biologically-resorbable cement.

### BACKGROUND

Bone grafts are commonly used in orthopedics as they provide key bone morphogenetic proteins (BMP) and support new bone growth. Allograft bone is considered osteoconductive and provides an excellent and important scaffold for new bone repair. This tissue can be further processed into demineralized bone matrix (DBM) through acid extraction, which removes the inorganic minerals from the allograft bone and leaves the organic collagen matrix. The demineralization process exposes osteoinductive growth factors and makes the graft a better promoter of new bone formation. However, the demineralization process also reduces the mechanical properties of the graft, so while the implanted graft is more biologically active, it does not provide adequate structural support to the surrounding bone.

To date, DBMs have been limited to morselized particles, granules, putties, gels, blocks and strips, which are typically placed manually in an open skeletal defect site or when in gel or putty form applied through a large gauge cannula/syringe. However, the majority of these particles are either too large to be injected and/or the graft is not cohesive enough to remain at the implanted site after injection. This makes DBMs unsuitable to treat bone defects confined within the cancellous bone, such as osteonecrosis, bone marrow edema, bone marrow lesions or other cysts or lytic lesions requiring a minimally invasive approach, unless the surgeon mechanically creates an open cavity or access channel in the cancellous bone through which the surgeons can apply the material. This technique can greatly compromise the stability of the bone, especially when used in weight bearing applications, and it also makes the procedure more invasive. Thus a bone graft that can be applied in a minimally invasively manner by means of injection through a needle or cannula would be highly beneficial to the orthopedic market.

With that in mind, it is appreciated that synthetic Bone Substitute Materials (BSMs), such as calcium sulfates, tri-calcium phosphates (TCPs) and calcium phosphates have been developed as an alternative to bone grafts and biologic BSMs. Particularly, self-setting calcium phosphate cements (CPCs) have long been used in orthopedic applications because the crystalline structures of those cements are similar to the mineral phase of natural bone, hydroxyapatite. This similarity lends itself to sufficient biocompatibility, as the release of calcium and phosphorus ions regulates the activation of osteoblasts and osteoclasts to facilitate bone regeneration. The self-setting nature of the material can also provide mechanical support to the surrounding tissue and prevent the material from migrating from the intended application site. The material's surface properties and porosity further provide a suitable scaffold for cell/protein adhesion and growth, and, as a result, the CPC is often slowly resorbed and remodeled by the body as new bone formation occurs in a process called creeping substitution.

While CPCs are highly biocompatible, can provide structural support to deteriorated bone, and can facilitate bone remodeling, the slow resorption properties of CPCs mean that portions of the implant can remain in the body for several years, and in some cases indefinitely. When a bolus of cement is injected into a diseased area of cancellous bone, it can positively disrupt the pathology in the short term. However, the slow resorption rate of synthetic only CPCs can be detrimental to the blood supply of the bone, which can lead to future complications and prevent the bone tissue from fully healing. This can be especially problematic in smaller bones, such as the talus, where the blood supply is already limited. Synthetic cements therefore are generally regarded as being for use in larger, well-vascularized applications, such as the knee, and restricted from use in more confined anatomical locations. Similarly, fully synthetic cements are undesirable for use in lesions such as avascular necrosis (AVN), which involve altered or diminished blood supply and necrotic tissue. On the other hand, other biologically-resorbable cements, such as calcium sulfate cements, set, but quickly dissolve and do not provide mechanical support.

Biologically-resorbable cements themselves are generally comprised of a mixture of powder and liquid. These materials are normally mixed in the operating room, molded and placed into a bone defect and then left to harden, usually within several minutes, making it a suitable bone graft substitute. The powder portion is often comprised of reactive particles of various sizes that, when combined with an appropriate amount of aqueous solution, form a paste-like consistency and can generally be considered as “flowable” before it crystallizes and hardens in situ. However, when a typical biologically-resorbable cement is injected through a syringe, the high pressure gradient between the syringe piston and exit point causes the liquid phase to travel at a faster rate than the particles and the liquid begins to filter through the network of particles. As a result, the extruded material has a higher liquid content than the material remaining in the syringe. This causes problems with the extruded material having altered properties and increases risk of extravasation from the surgical site and of the possibility of embolism if taken up by the vasculature.

Additionally, the material in the syringe often has reduced liquid content and becomes dry, compacted and un-extrudable. This phenomenon is generally referred to as ‘pressure filtration’ or ‘phase separation’ and limits most biologically-resorbable cements from being injected through a small cannula/needle or into a confined space with low porosity, such as relatively high density cancellous bone at the proximal and distal ends of the long bone in the extremities or in the cancellous bone within the pelvis, spine, and other regions of the skeleton. Consequently, this behavior restricts their use in minimally invasive surgical applications, with this phenomenon also being applicable to any biomaterial comprising particles suspended in a liquid. In order to be effectively injectable into high resistance spaces the BSM's rheological properties should allow injection of the cement through a cannula/needle into the skeletal defect with a force that can be comfortably applied by the hand of an orthopedic surgeon (approximately 100N). At the same time, the extruded BSM should also have sufficient cohesion to remain at the implant site and avoid dispersion in the body. To date, only a limited number of CPCs have achieved this level of injectability and an injectable bone graft has yet to be produced.

Many of the conditions or defects within cancellous bone that need to be addressed require a treatment solution (graft or graft substitute) with several desirable properties that are not always simultaneously achievable. First, the graft or graft substitute should be able to be delivered in a minimally invasive manner, which would typically be by injection through a relatively small cannula or needle. Second, the graft or graft substitute should provide immediate mechanical support and structural stabilization. Third, the material should not migrate from the location into which it is placed. And finally, the graft or graft substitute should remodel quickly into healthy living bone tissue for healing and resolution of the condition or defect.

Accordingly, a bone graft that can be applied using minimally invasive techniques and that can remodel completely into host bone without disrupting and/or preventing the bone tissue from fully healing would dramatically improve treatment for cancellous bone disease. Moreover, an improved bone implant composition that makes use of and combines the beneficial effects of both biologically-resorbable cements and bone particles, while still maintaining the injectability of such an implant composition would be both highly desirable and beneficial.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

The presently-disclosed subject matter includes injectable bone implant compositions, kits, and methods for making and using the bone implant compositions. In particular, certain embodiments of the presently-disclosed subject matter relate to injectable bone implant compositions and related kits and methods that make use of an injectable bone graft comprising a plurality of bone microparticles and a viscous binder, where the plurality of bone microparticles have a size and, in certain embodiments, a level of demineralization that improves the injectability of the bone implant compositions.

In some embodiments, each of the bone microparticles have a particle size of less than about 250 μm, such as, in certain embodiments, a particle size of less than about 212 μm. In some embodiments, the plurality of bone microparticles included in the bone implant composition has a mean particle size of less than about 45 μm such as, in some embodiments, a mean particle size of less than about 35 μm. In other embodiments, the plurality of bone microparticles included in an exemplary composition can be described by or otherwise defined with reference to the particle size distribution of the bone microparticles included in an exemplary composition. In some embodiments, the plurality of bone microparticles has a particle size distribution such that about ten percent of the bone microparticles have a particle size of about 10 μm or less. In some embodiments, the plurality of bone microparticles has a particle size distribution such that about fifty percent of the bone microparticles have a particle size of about 70 μm or less, about 30 μm or less, about 20 μm or less, or about 5 μm or less. In some embodiments, the plurality of bone microparticles has a particle size distribution such that about ninety percent of the bone microparticles have a particle size of about 100 μm or less, about 75 μm or less, or about 50 μm or less. In some embodiments, the plurality of bone microparticles have a particle size distribution such that about ninety percent of the bone microparticles have a particle size of about 30 μm or less.

With further regard to the bone microparticles included in an exemplary bone implant composition of the presently-disclosed subject matter, in some embodiments, the bone microparticles comprise cortical bone, cancellous bone, or both cortical and cancellous bone. In some embodiments, the bone microparticles comprise cortical bone. Further, in some embodiments, the bone microparticles are comprised of allograft bone microparticles, autograft bone microparticles, xenograft bone microparticles, or combinations thereof. In some embodiments, the bone microparticles comprise allograft bone microparticles.

The bone microparticles, as indicated above, can, in certain embodiments, also have a level of demineralization that allows the bone implant compositions to be more readily incorporated, remodeled, and resorbed into a subject, while also allowing for a bone implant composition to be provided having an improved injectability. In some embodiments, the bone microparticles are at least partially demineralized such as, in some embodiments, about 5% to about 95% demineralized. In some embodiments, an amount of demineralization of each of the bone microparticles is different. In other embodiments, however, the bone microparticles are fully mineralized or the bone microparticles are comprised of a mixture of mineralized bone microparticles and at least partially demineralized bone microparticles.

In some embodiments, as also indicated above, the bone microparticles are further combined with one or more additional components to produce a bone implant composition in accordance with the presently-disclosed subject matter. In some embodiments, the viscous binder that is combined with the bone microparticles comprises cellulose such as, in certain embodiments, sodium carboxymethyl cellulose.

In some embodiments, the bone implant compositions further comprise a biologically-resorbable cement including, in some embodiments, a calcium-based cement such as a calcium phosphate cement or a calcium sulfate cement. In some embodiments that make use of a calcium-based cement, the calcium-based cement is a calcium phosphate cement that has a calcium:phosphate ratio of about 1.67 in a powder phase. In some embodiments, the calcium-based cement is a calcium phosphate cement that is comprised of calcium phosphate particles having a particle size of less than about 150 μm. In some embodiments, the biologically-resorbable cement is in the form of a powder that comprises about 40 wt. % to about 90 wt. % of a combined amount of the biologically-resorbable cement and the bone microparticles. In some embodiments, the bone microparticles comprise about 10 wt. % to about 60 wt. % of a combined amount of the biologically-resorbable cement and the bone microparticles. In certain embodiments of the presently-disclosed subject matter, the biologically-resorbable cement comprises a resorbable ceramic powder. In some embodiments, rather than making use of a biologically-resorbable cement, the compositions make use of a synthetic bone graft extender, such as calcium phosphate granules.

Additionally included in some bone implant compositions of the presently-disclosed subject matter is a hydration liquid. In certain embodiments, the hydration liquid is included in a particular composition relative to the amount of viscous binder or biologically-resorbable cement included in the composition. For instance, in some embodiments, the viscous binder comprises about 0.1 wt. % to about 15 wt. % of the hydration liquid. As another example, in some embodiments that make use of a biologically-resorbable cement, the hydration liquid comprises about 35 wt. % to about 170 wt. % of a combined amount of the biologically-resorbable cement and bone microparticles included in the composition.

With further regard to the hydration liquid, in some embodiments, the hydration liquid comprises a phosphate solution, a biological fluid, a saline solution, or water. For instance, in some embodiments, the hydration liquid is a phosphate solution, such as a sodium phosphate dibasic solution. In some embodiments, the hydration liquid is a biological fluid, including, for example, a biological fluid selected from the group consisting of blood, plasma, blood serum, platelet rich plasma, bone marrow aspirate (BMA), or bone marrow aspirate concentration (BMAC).

In some embodiments of the presently-described bone implant compositions, an exemplary bone implant composition is provided that is not a cement per se in that it does not set and harden, but is nonetheless provided as an injectable composition capable of use as a bone graft composition. In some embodiments, such a composition is comprised of a plurality of the bone microparticles described herein and having various levels of demineralization, which is then combined with a viscous binder to produce an injectable composition. In some embodiments, such a non-setting injectable composition further includes an amount of calcium phosphate (non-cement) such as an amount of calcium phosphate microparticles.

In some embodiments of the bone implant compositions described herein, the bone implant compositions can be provided in a manner that divides the compositions into: a powder component including a biologically-resorbable cement; and a liquid portion that includes the hydration liquid combined with a plurality of the bone microparticles and the viscous binder. In some of these embodiments, the biologically-resorbable cement is a calcium-based cement powder, such as a calcium phosphate cement or a calcium sulfate cement. In some embodiments that make use of separate powder and liquid components, the hydration liquid comprises about 35 wt. % to about 170 wt. % of a combined amount of the biologically-resorbable cement and the bone microparticles. In some such embodiments, the bone microparticles comprise about 11 wt. % to about 150 wt. % of the biologically-resorbable cement and, in certain of embodiments, the viscous binder comprises about 0.1 wt. % to about 15 wt. % of the hydration liquid.

Further provided in some embodiments of the presently-disclosed subject matter are kits that include a bone implant composition as described herein. In some embodiments, a kit is provided that comprises a biologically-resorbable cement powder, a viscous binder, and a plurality of bone microparticles, where each of the bone microparticles have a particle size of less than about 250 μm. In some kits, the kits further comprise a hydration liquid such that, in certain embodiments, the biologically-resorbable cement powder and the plurality of bone microparticles are included in a first container, and the hydration liquid is included in a second container. In other embodiments, the biologically-resorbable cement powder is included in a first container, and the hydration liquid and the plurality of bone microparticles are included in a second container. In some embodiments, the kits further comprise instructions for mixing the hydration liquid with the biologically-resorbable cement powder, the viscous binder, and/or the plurality of bone microparticles.

Additionally provided, in some embodiments of an exemplary kit, are various instruments and other means for delivery the exemplary bone implant compositions. In some embodiments, for example, a kit further comprises a delivery cannula for delivery of the bone implant composition. In some embodiments, a kit is provided that further comprises a syringe for mixing or delivering the bone implant composition.

Still further provided in some embodiments of the presently-disclosed subject matter are methods for treating a bone defect whereby an effective amount of a bone implant composition is administered to a site of a bone defect in a subject. In some embodiments, the defect is a bone void, a fracture, osteonecrosis, bone edema, a bone marrow lesion, a cyst or a lytic lesion. In some embodiments, administering the bone implant composition comprises administering the bone implant composition to augment placement of bone hardware in the subject. In other embodiments, administering the bone implant composition comprises administering the bone implant composition through a syringe or cannula.

To administer an exemplary bone implant composition described herein, in some embodiments, administering the composition comprises applying a force of less than 100 N to the syringe or cannula to inject the bone implant composition into a bone. In some embodiments, administering the bone implant composition comprises filling the bone defect with the bone implant composition. To facilitate the administration of an exemplary bone implant composition, in some embodiments, the therapeutic methods further comprise a step of imaging the site of the bone defect in the subject prior to administering the bone implant composition, such as, for example, by imaging the site of the bone defect with x-ray, computed tomography (CT), and/or magnetic resonance imaging (MRI).

Even further provided, in certain embodiments of the presently-disclosed subject matter are methods of making a bone implant composition. In some embodiments, an exemplary method of making a bone implant composition includes an initial step of obtaining an amount of intact bone. The intact bone is then milled to produce a plurality of bone microparticles, and the plurality of bone microparticles are then demineralized by exposing the plurality of bone microparticles to an amount of acid and then neutralizing the acid. After the bone microparticles are exposed to an amount of acid and after neutralizing the acid, however, the bone microparticles undergo a centrifugation step that allow any liquid to be removed (e.g., decanted or poured off) from the bone microparticles in a timely manner, but also allows such liquid to be removed without causing any significant loss of the bone microparticles. In some embodiments, exposing the bone microparticles to an amount of acid comprises exposing the bone microparticles to an amount of acid calculated to remove a predetermined amount of calcium from the plurality of bone microparticles. In some embodiments of the manufacturing methods, the methods further comprise a step of rinsing the plurality of bone microparticles prior to exposing the bone microparticles to an amount of acid and subsequent to neutralizing the acid. In some embodiments, subsequent to demineralization, the bone microparticles are lyophilized.

Further features and advantages of the present invention will become evident to those of ordinary skill in the art after a study of the description, figures, and non-limiting examples in this document.

### BRIEF DESCRIPTION OF THE DRAWINGS

FIGS. 1A-IC include schematic diagrams showing a disruption in the cancellous portion of a bone (FIG. 1A), injection of a bioactive bone implant composition of the presently-disclosed subject matter into the defect (FIG. 1B), and remodeling of the defect into healthy native bone overtime (FIG. 1C);

FIGS. 2A-2E show a delivery cannula for use in injecting a bone implant composition in accordance with the presently-disclosed subject matter;

FIG. 3 is a graph illustrating the demineralization process utilized to produce exemplary bone microparticles in accordance with the presently-disclosed subject matter, and showing the pH of a composition as a function of time during exposure of the bone microparticles to an amount of acid; and

FIG. 4 is a graph showing the linear relationship between the ratio of acid to bone microparticles and the residual calcium content.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended), “consist of” (closed ended), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

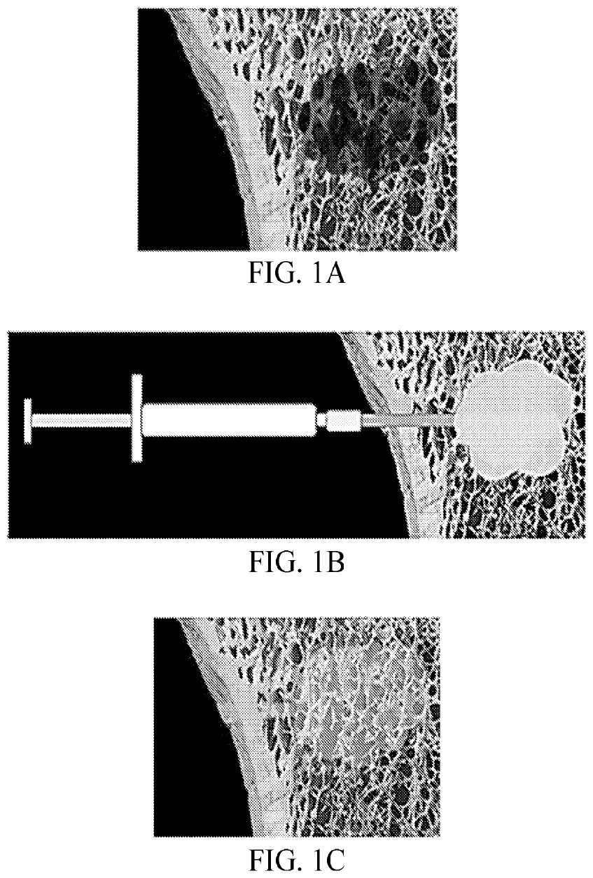

The presently-disclosed subject matter is based, at least in part, on the discovery and development of an injectable bone graft substitute or implant composition that makes use of a viscous binder in combination with a plurality of bone microparticles to increase the viscosity of the composition and keep the bone microparticles in suspension. In some embodiments, the use of a such a viscous binder reduces the ability of any liquid to flow between the particles and instead keeps the particles and liquid flowing together as a cohesive fluid. Moreover, in some embodiments, by further including and making use of a biologically-resorbable cement, such as a calcium phosphate cement, that has an appropriate particle size, distribution and shape, in connection with an appropriate amount and concentration of a hydration liquid and the viscous binder, it has been observed that phase separation is significantly reduced or eliminated, but that the material still retains appropriate handling, setting, and mechanical properties. In particular, in some embodiments, adding such a viscous binder to the biologically-resorbable cement does not disrupt the end crystalline structure or the biocompatibility of the cement, yet allows the cement to be injected into the most challenging of bone sites (see, e.g., FIGS. 1A-1C).

In some embodiments of the presently-disclosed subject matter, bone implant compositions are provided that comprise a plurality of bone microparticles and a viscous binder. In some embodiments, in such a bone implant composition, each of the bone microparticles have a particle size of less than about 250 μm. In some embodiments, the bone implant composition further comprises a biologically-resorbable cement and/or a hydration liquid, as described in further detail below.

With regard to the processed bone particles that are included in the presently-disclosed bone graft compositions, the phrase “bone microparticles” is used herein to refer to pieces of bone that are derived from an intact bone, or part of an intact bone, and have been modified to produce pieces of bone with a desired level of mineralization and a desired size, such that the pieces of bone can be combined with viscous binder and/or a suitable cement and applied to the site of a bone defect, as described in detail below. In some embodiments, the bone microparticles are of a size that allows a prescribed mixture of cement (e.g., calcium phosphate cement), a viscous binder, and/or bone microparticles to be combined with a hydration liquid and flow in a manner similar to a cement by itself, while still keeping the bone microparticles in suspension. In some embodiments, the bone microparticles have a particle size of about 0.01 μm to about 250 μm. In some embodiments, the processed bone particles are from an autograft bone source, an allograft bone source, a xenograft bone source, or combinations thereof.

In some embodiments, each of the bone microparticles have a particle size of less than about 250 μm, such as, in certain embodiments, a particle size of less than about 212 μm, which are obtained through the use of a milling and sieving procedure as described in further detail below. In some embodiments, the plurality of bone microparticles included in the bone implant composition has a particle size ranging from 1 μm to about 250 μm. In some embodiments, the plurality of bone microparticles included in the bone implant composition has a mean particle size of less than about 45 μm such as, in some embodiments, a mean particle size of less than about 35 μm. In other embodiments, the plurality of bone microparticles included in an exemplary composition can be described by or otherwise defined with reference to the particle size distribution of the bone microparticles included in an exemplary composition. In some embodiments, the plurality of bone microparticles has a particle size distribution such that about ten percent of the bone microparticles have a particle size of about 10 μm or less. In some embodiments, the plurality of bone microparticles has a particle size distribution such that about fifty percent of the bone microparticles have a particle size of about 70 μm or less, about 30 μm or less, about 20 μm or less, or about 5 μm or less. In some embodiments, the plurality of bone microparticles has a particle size distribution such that about ninety percent of the bone microparticles have a particle size of about 100 μm or less, about 75 μm or less, or about 50 μm or less. In some embodiments, the plurality of bone microparticles have a particle size distribution such that about ninety percent of the bone microparticles have a particle size of about 30 μm or less.

To produce a plurality of bone microparticles in accordance with the presently-disclosed subject matter and having such above-described particle sizes, an intact bone is typically first obtained from one of the sources indicated above and is refined into a number of portions capable of being further processed. The term “intact bone” is used herein to refer to whole bones or segments of whole bones whose structures have not been substantially altered, broken, or impaired prior to being used to produce a bone particle in accordance with the presently-disclosed subject matter. In some embodiments, the intact bone used to produce the bone microparticles is selected from a tibia, fibula, femur, humerus, radius, ulna, rib bone, or portions thereof, and can include cortical and/or cancellous bone.

Upon processing the bone into a number of smaller portions, any debris, soft tissue, blood, bone marrow, and/or lipids are first removed from the bone and the bone is subsequently dried or freeze dried. The dried portions of bone are then typically further reduced to a distribution of particles having a desired size. In this regard, the term “particle size” is used herein to refer to particles of bone parties having a diameter or longest dimension of a desired size. For instance, reference to a bone microparticle having a particle size of less than 212 μm refers to bone microparticles whose diameter or longest dimension is less than 212 μm. Of course, the size and shape of the bone microparticles will depend, at least in part, on the procedure utilized to produce the bone microparticles. In some embodiments, however, such bone microparticles are produced through an abrasive grinding or rotating blade milling procedure, which is performed while maintaining a low temperature (e.g., <40° C.) so as not to denature the tissue or proteins within the bone. In some embodiments, such a milling procedure is performed through the use of a centrifugal mill, such as a Fritsch Pulverisette 14 (Fritsch Milling and Sizing Inc., Pittsboro, NC).

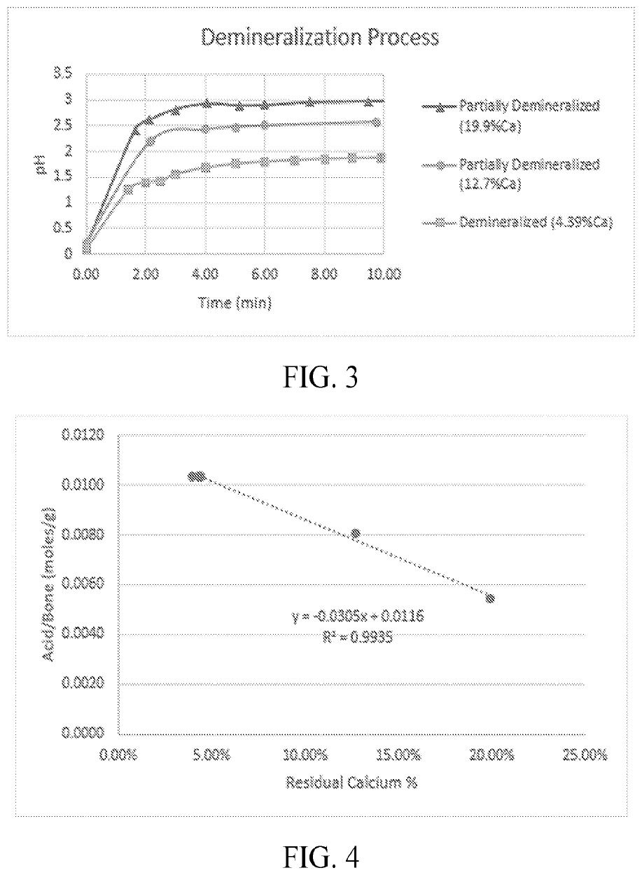

Upon producing the microparticles by the milling procedure, the fine particles (e.g., less than 250 μm) can then be captured through a controlled screening (e.g., sieving) procedure or similar process to insure each batch of particles is below a defined and predetermined particle size. Once the fine particles are captured, the particles are then typically washed and demineralized, as described in further detail below, in a series of soaking, agitating, and centrifugation procedures involving alcohol, hydrochloric acid (or other acids such as EDTA, citric acid, or lactic acid), and neutralization agents, followed by further rinsing steps before a final centrifugation. In particular, in some embodiments of the presently-disclosed subject matter, the bone microparticles are chemically processed by first rising the bone microparticles with a rinsing agent, such as ethanol or another alcohol, to further clean the bone microparticles, while also killing any microorganisms, bacteria, spores, or the like. The rinsed bone microparticles are then exposed to an acid bath to demineralize and remove the calcium from the bone microparticles in a controlled manner and in a manner that further exposes the bone morphogenetic proteins (BMPs) present in the bone microparticles. In some embodiments, rather than relying of timing to control the demineralization reaction during the exposure to the acid, the quantity or amount (e.g., number of moles) of acid needed to remove a predetermined amount of calcium is calculated and that amount of acid is then used to control the amount of calcium that is removed from the bone microparticles. In this way, in some embodiments, the acid input controls the level of demineralization of the bone microparticles (i.e. the resulting calcium content) in a surprisingly repeatable manner and in a manner that is not dependent on the time of exposure of the bone microparticles to the acid. In some implementations, the acid used to demineralize the bone microparticles is further selected and configured so as to provide the acid at an appropriate concentration and pH that allows demineralization, but does not cause unnecessary destruction or denaturing of the proteins and the collagen (i.e., organic) portions of the bone tissue. For example, to demineralize bone microparticles to a desired partially demineralized level of 12.70% residual calcium content, an acid to bone ratio of 0.0077 is used (See, e.g., FIG. 4), such that, for processing 30 grams of bone microparticles, 0.231 moles of acid (231 mL of 1M HCl) is utilized. Similarly, and as another example, for a desired demineralized level of 4.39% calcium, an acid to bone ratio of 0.0103 is used, requiring 0.309 moles of acid. In some implementations, the timing of the acid exposure to the bone tissue is further utilized but, as indicated above, is not necessarily required so as to allow demineralization but not cause destruction or denaturing of the proteins and the collagen portions of the bone tissue.

Following exposure to the acid, in some embodiments, a neutralization agent such as phosphate buffered saline (PBS), a potassium phosphate buffer (KPB) sodium bicarbonate (Na₂CO₃) solution, sodium hydroxide (NaOH), Sorensen's Buffer, or the like, is then used to neutralize any residual acid and bring the pH of the bone microparticles back to neutral (e.g., a pH of about 7). In some embodiments, the neutralization step is followed by a further rinsing step, such as with water, to dilute any residual chemicals as it is believed that proper neutralization and rinsing of the tissue after the acid exposure is important to maintain stability of the proteins and collagen and ensure biocompatibility of the compositions with the cells in a subject. As the bone microparticles proceed through each step, however, and after exposing the bone microparticles to an amount of acid, neutralizing the bone microparticles, or rinsing the bone microparticles, a centrifuge is used to force the particles out of liquid suspension and compact the particles into a plug of material at the bottom of a container (e.g., a centrifuge tube) containing the suspension. Centrifuging the bone microparticles in this manner allows any liquid to be removed (e.g., decanted or poured off) from the bone microparticles in a timely manner, but further allows such liquid to be removed without causing any significant loss of the yield of bone microparticles and without causing overexposure of the bone microparticles to the acid or neutralization agent.

Following demineralization and rinsing, the bone microparticles described herein are then rewetted with either water or an alcohol solution to be prepared for preservation or storage. Drying is subsequently performed in a desiccating oven and/or a lyophilizer to produce dried demineralized (partial or combo) bone microparticles. In some embodiments, by making use of the above-described process, the microparticles have a reproducible average mass, which allows the bone microparticles to be used for acceptable mass combination calculations when combining components into kits, and which allows the bone microparticles to be easily mixed with the calcium phosphate (or similar) biologically-resorbable cement powders along with any other components and a hydration liquid to form a final biological implant composition or product.

In some embodiments, the bone microparticles used in accordance with the present compositions are comprised of bone tissue derived from cortical bone, cancellous bone, or both cortical and cancellous bone. In some embodiments, as indicated above and irrespective of whether cortical or cancellous bone is used as a starting material, the produced bone microparticles (e.g., as produced by the milling and grinding procedure described above) are demineralized to provide a means to facilitate the movement of cells and fluids to the interior of the bone graft. The term “demineralized” is used herein to refer to the process by which bone mineral or the inorganic portion of the bone is removed to thereby expose the collagen matrix portion of the bone. In this regard, in some embodiments and as also indicated above, to prepare a bone microparticle of the presently-disclosed subject matter (e.g., a bone microparticle derived from cortical bone), a demineralization process can be used such that the outer surface of the bone microparticle is transformed into an exposed collagen layer that is then capable of stimulating and facilitating the infiltration and activity of cells and fluid from the subject (e.g., the environments surrounding the grafted site) into the bone graft. In some embodiments, the bone microparticles particles are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, to about 100% demineralized. In some embodiments, 0% demineralized constitutes fully mineralized bone tissue, containing the calcium content originally found in the bone microparticles (i.e. before demineralization) and 100% demineralized constitutes bone microparticles with no residual calcium content. In some embodiments, and as used herein, calcium content is assessed according to the American Association of Tissue Banks (AATB) standards for tissue banking, where residual calcium content for bone labeled as demineralized does not exceed 8% as determined by standard methodology, whereas for bone that has been subjected to a demineralization process with a residual calcium content target that exceeds 8% when tested, such bone is labeled as partially demineralized to describe the extent of demineralization.

In some embodiments, by demineralizing the bone particles, the speed with which the bone implant composition is incorporated, remodeled, or resorbed into the subject and replaced by living bone is increased, while the bone implant composition maintains and improves the strength of the subject's bone and the composition itself. In some embodiments, if the processed bone particles are not from an autograft source, the demineralization of the processed bone microparticles can increase the rate at which the bone implant composition is incorporated into the subject and replaced with living bone from the subject. In some embodiments of the presently-described bone implant compositions, the bone microparticles are at least partially demineralized, are fully mineralized, or are comprised of a mixture of mineralized bone microparticles and at least partially demineralized bone microparticles.

Turning now to the viscous binders included in an exemplary bone implant composition, the term “viscous binder” is used herein to refer to materials capable of holding or drawing other materials together to form a cohesive whole mechanically and/or chemically such as by viscosification and/or cohesion. In this regard, in connection with the presently-described bone implant compositions, the term “viscous binder” is thus inclusive of materials that are capable of being included in an amount sufficient to improve the handling and injection properties of an exemplary bone implant composition, including a reduction in phase separation. Viscous binders capable of use in accordance with the presently-disclosed subject matter include, but are not limited to, celluloses (methylcellulose, carboxy methylcellulose, hydroxypropyl methylcellulose, hydroxyethyl cellulose), carrageenan, starches, gelatins, gums (xanthan, arabic, guar), glucose, glycogens, dextrans, collagen, glycerol, glycerine, polymers and biopolymers, polysaccharides, proteins, carbohydrates, chitin, chitosans, carbomers, agar, hyaluronic acid, sodium hyaluronate, and mixtures thereof. In some embodiments, the viscous binder comprises cellulose. In some embodiments, the viscous binder comprises sodium carboxymethyl cellulose.

In some embodiments, the viscous binder can comprise glycerin or corn starch. In some embodiments, the viscous binders added to the compositions are added in an amount sufficient to maintain biocompatibility of the implant composition, while preserving the osteoconductive or osteoinductive nature of the bone microparticles. In each composition, however, the viscous binders are typically added in sufficient quantity to produce an injectability profile whereby 100% of the material can be extruded from a delivery device (e.g., a cannula or syringe) to the site of a bone defect using a force less than or equal to 100 N, such as, in certain embodiments, a force of about 100 N, about 95 N, about 90 N, about 85 N, about 80 N, about 75 N, about 70 N, about 65 N, about 60 N, about 55 N, or about 50 N.

In some further embodiments of the presently-disclosed bone implant compositions, and as indicated above, a bone implant composition is provided that further includes a biologically-resorbable cement. The term “biologically-resorbable cement” is, in certain instances, used interchangeably with the term “biologically-resorbable cement powder” and is used herein to refer to any biological cement, such as a bone substitute cement, that is capable of being broken down and assimilated by the body of a subject, and that is substantially non-toxic in the in vivo environment of its intended use, such that it is not substantially rejected by the subject's physiological system (i.e., is non-antigenic or biocompatible). This can be gauged by the composition's toxicity, infectivity, pyrogenicity, irritation potential, reactivity, hemolytic activity, carcinogenicity and/or immunogenicity. A biologically-resorbable cement, when introduced into a bone of a majority of subjects, will not cause an undesirably adverse, long-lived or escalating biological reaction or response, and is distinguished from a mild, transient inflammation which typically accompanies surgery or implantation of foreign objects into a living organism.

As would be recognized by those skilled in the art, a “cement” is a product that is produced as a result of the setting of a paste that is formed by mixing a powdered component with a hydration liquid, such as water or another aqueous vehicle. A number of biologically-resorbable cements can be formed by mixing a powder component with a hydration liquid and then used in accordance with the presently-disclosed bone implant compositions, including, but not limited to, ceramics-based cements, calcium-based cements, magnesium phosphate- or ammonium-based cements, and the like. In some embodiments of the presently-disclosed compositions, the biologically-resorbable cement is a calcium-based cement, such as a calcium sulfate cement or a calcium phosphate cement, where the powdered component is comprised of a calcium-based compound. In some embodiments, the calcium-based cement is a calcium phosphate cement. In other embodiments, the calcium-based cement is a calcium sulfate cement.

The phrase “calcium phosphate cement” is used herein to refer to a cement where the powdered component of the cement is comprised of a calcium phosphate compound or a mixture of calcium and/or phosphate compounds. Exemplary calcium phosphate compounds or mixtures of calcium compounds and/or phosphate compounds that can be mixed with water or another aqueous vehicle and used in accordance with the presently-disclosed subject matter include, but are not limited to: tricalcium phosphate (Ca₃(PO₄)₂; TCP), including alpha-TCP, beta-TCP, and biphasic calcium phosphate containing alpha- and beta-TCP; amorphous calcium phosphate (ACP); calcium oxide (CaO); monocalcium phosphate (Ca(H₂PO₄)₂; MCP) and monocalcium phosphate monohydrate (Ca(H₂PO₄)₂·H₂O; MCPM); dicalcium phosphate (CaHPO₄; DCP), dicalcium phosphate anhydrous (CaHPO₄; DCPA) and dicalcium phosphate dihydrate (CaH₅PO₆·2H₂O; DCPD); tetracalcium phosphate ((Ca₄PO₄)₂O; TTCP); octacalcium phosphate (Ca₈(PO₄)₄HPO₄)₂·5H₂O; OCP); calcium hydroxyapatite (Ca₁₀(PO₄)₆(OH)₂; CHA); calcium oxyapatite (Ca₁₀(PO₄)₆O; COXA); calcium carbonate (CaCO₃); calcium carbonate apatite (Ca₁₀(PO₄)₆CO₃; CCA); and calcium carbonate hydroxyapatites (e.g., Ca₁₀(PO₄)₅(OH)(CO₃)₂ and Ca₁₀(PO₄)₄(OH)₂(CO₃)₃; CCHA). Additional calcium phosphates useful herein also include calcium-deficient calcium phosphates in which the molar or mass ratio of Ca:P is reduced by about 20% or less, about 15% or less, or about 10% or less, relative to the corresponding calcium non-deficient species, examples of which include calcium-deficient hydroxyapatites, e.g., Ca₁₀₋ₓ(HPO₄)ₓ(PO₄)₆₋ₓ(OH)_{2-X}(O≤X≤1) (CDHA); calcium-deficient carbonate hydroxyapatites (CDCHA); calcium-deficient carbonate apatites (CDCA); and other calcium phosphate compounds and salts known to be useful in the field of bone graft materials, e.g., calcium polyphosphates; and calcium-, phosphate-, and/or hydroxyl “replaced” calcium phosphates. In some embodiments, the calcium-based cement is a calcium phosphate cement that has a calcium:phosphate ratio of about 1.67 in a powder phase. In some embodiments, the calcium-based cement is a calcium phosphate cement formed from a calcium phosphate cement powder having calcium phosphate particles with a particle size of less than about 150 μm. For further explanation and guidance regarding calcium phosphate cements, see, e.g., Ambard, et al. Journal of Prosthodontics. 15(5): 321-326 (2006).

The phrase “calcium sulfate cement” is used herein to refer to a cement where the powdered component of the cement is comprised of a calcium sulfate compound or a mixture of calcium and/or sulfate compounds. Exemplary calcium sulfate compounds or mixtures of calcium compounds and/or sulfate compounds that can be mixed with water or another aqueous vehicle and used in accordance with the presently-disclosed subject matter include, but are not limited to: calcium sulfate (CaSO₄); calcium sulfate dihydrate (2CaSO₄·2H₂O); and calcium sulfate hemihydrate (CaSO₄·½ H₂O). For further explanation and guidance regarding calcium sulfate cements, see, e.g., Bohner, European Cells & Materials, Vol. 20, 2010, pages 1-12.

In some embodiments of the presently-disclosed bone implant compositions, the biologically-resorbable cement comprises a resorbable ceramic powder. In some embodiments, such a resorbable ceramic powder comprises a calcium or magnesium phosphate or sulfate powder. In some embodiments, the biologically-resorbable cement comprises a magnesium phosphate cement. In some embodiments, a bone implant composition is provided that comprises a plurality of bone microparticles with each of the bone microparticles having a particle size of less than about 250 μm, a viscous binder, and a biologically-resorbable ceramic powder. In some embodiments, a bone implant composition is provided that comprises a plurality of bone microparticles with each of the bone microparticles having a particle size of less than about 250 μm, a viscous binder, and a synthetic bone graft extender. In some embodiments, such a synthetic bone graft extender comprises calcium phosphate granules.

With regard to the hydration liquid included in an exemplary bone particle composition, a number of suitable aqueous vehicles can be selected to be combined with the bone microparticles, viscous binder, and biologically-resorbable cement powder to form a bone implant composition in accordance with the presently-disclosed subject matter. In some embodiments, the hydration liquid comprises a phosphate solution, a biological fluid, a saline solution, or water. In some embodiments in which a phosphate solution is utilized, the phosphate solution is a sodium phosphate dibasic solution. In other embodiments in which a biological fluid is utilized, the biological fluid is selected from the group consisting of whole blood, blood plasma or serum, platelet rich plasma, bone marrow aspirate (BMA), and bone marrow aspirate concentrate (BMAC).

To produce an exemplary bone implant material or composition in accordance with the presently-disclosed subject matter, in some embodiments, the bone microparticles can be combined first with the biologically-resorbable cement powder to form a powder component that can then be combined with the hydration liquid. For example, in some embodiments, a bone implant composition is produced by a process in which a powder component is first formed where a biologically-resorbable cement powder is included in the powder component in an amount of about 40 wt. % to about 90 wt. % of the powder component, and where the bone microparticles are included in an amount of about 10 wt. % to about 60 wt. % of the powder component. That powder component including the combination of biologically-resorbable cement powder is then mixed with a suitable hydration liquid where the hydration liquid is included in an amount that is about 35 wt. % to about 170 wt. % of the powder component. In such embodiments, the viscous binder can be included in either the powder component or the liquid component used for the bone implant composition, but is generally included in an amount that is about 0.1 wt. % to about 15 wt. % of the hydration liquid.

As another example of how a bone implant material can be produced in accordance with the presently-disclosed subject matter, in some embodiments, a bone implant composition is produced by a process in which the biologically-resorbable cement powder is used by itself to form a powder component that can then be combined with a hydration liquid, where the hydration liquid has already been combined with a plurality of bone microparticles. In such an embodiment, a bone implant composition is produced by a process in which the powder component initially includes any amount of a biologically-resorbable cement powder. In such an embodiment, the liquid component is then formed by combining the hydration liquid with a plurality of bone microparticles, where the bone microparticles are included in an amount that is about 11 wt. % to about 150 wt. %. of the amount of cement powder, and where the hydration liquid is then included in an amount that is about 35 wt. % to about 170 wt. % of the total mass of the cement powder and bone microparticles. Again, in such embodiments, the viscous binder can be included in either the powder component or the liquid component used to produce the bone implant composition, but is also generally included in an amount that is about 0.1 wt. % to about 15 wt. % of the hydration liquid.

In some embodiments of the presently-described bone implant compositions, an exemplary bone implant composition is provided that is not a cement per se in that it does not set and harden, but is nonetheless provided as an injectable composition capable of use as a bone graft composition. In some embodiments, such a composition is comprised of a plurality of the bone microparticles described herein and having various levels of demineralization, which is then combined with a viscous binder and a hydration liquid to produce an injectable composition. In some embodiments, such a non-setting injectable composition further includes an amount of calcium phosphate (non-cement) such as an amount of calcium phosphate microparticles. In some embodiments of such non-setting bone implant compositions, when the hydration liquid is included, the hydration is included in an amount that is about 35 wt. % to about 170 wt. % of the total mass of the bone microparticles. Again, in such embodiments, the viscous binder can be included in either the powder component or the liquid component used to produce the bone implant composition, but is also generally included in an amount that is about 0.1 wt. % to about 15 wt. % of the hydration liquid.

In addition to combining the components of the bone implant compositions outlined herein above and in the various amounts and ratios, in some embodiments, one or more further osteoinductive or osteogenic materials can also be included in an exemplary bone implant composition. In this regard, the term “osteoinductive material” is used herein to refer to any material that stimulates the migration or differentiation of bone cells to grow and become active at a graft site, while the term “osteogenic material” is used herein to refer to any material that is capable of directly or indirectly contributing to the action of osteoblasts or other cells capable of contributing to new bone growth. In some embodiments, the osteoinductive material that is added to an exemplary bone implant composition is selected from protein growth factors such as bone morphogenetic proteins (BMPs) and other proteins from the transforming growth factor-beta superfamily. In some embodiments, the osteogenic materials that can be added to the compositions include host cells (e.g., osteoblasts, etc.) or stem cells or progenitor cells.

In some embodiments, to add an osteoinductive and/or an osteogenic agent to the exposed surface of a bone microparticle (e.g., a partially demineralized bone microparticle having exposed collagen at its surface), the processed bone particles can be soaked in a solution containing the osteoinductive agent, the osteogenic agent, or both, prior to mixing the demineralized bone particles with the biologically-resorbable cement or other components of an exemplary composition, such that the osteoinductive and/or osteogenic agent simply incorporates into and adheres to the bone microparticle itself. In some other embodiments, the osteoinductive agent, the osteogenic agent, or both can be absorbed to the bone microparticles after mixing the demineralized bone particles with the biologically-resorbable cement or other components of an exemplary composition, or may be incorporated onto the surface of the bone microparticles by virtue of the placement of the bone microparticles within a subject. Of course, a number of other methods for linking such an agent to a protein such as collagen are known to those of ordinary skill in the art and can be used without departing from the spirit and scope of the subject matter described herein.

In some embodiments, stem cells can further be added to the bone implant compositions to enhance the incorporation of the bone implant composition into the subject and its replacement with living bone from the subject. As used herein, the term “stem cells” refers broadly to traditional stem cells, progenitor cells, preprogenitor cells, precursor cells, blood cells, platelets, reserve cells, and the like. Exemplary stem cells include, but are not limited to, embryonic stem cells, adult stem cells, pluripotent stem cells, neural stem cells, muscle stem cells, muscle precursor stem cells, endothelial progenitor cells, bone marrow stem cells, chondrogenic stem cells, lymphoid stem cells, mesenchymal stem cells, hematopoietic stem cells, and the like. Descriptions of stem cells, including methods for isolating and culturing them, may be found in, among other places, Embryonic Stem Cells, Methods and Protocols, Turksen, ed., Humana Press, 2002; Weisman et al., Annu. Rev. Cell. Dev. Biol. 17:387-403; Pittinger et al., Science, 284:143-47, 1999; Animal Cell Culture, Masters, ed., Oxford University Press, 2000; Jackson et al., PNAS 96(25):14482-86, 1999; Zuk et al., Tissue Engineering, 7:211-228, 2001; Shi, et al., Tissue Engineering Part A, 18(13-14): 1313-21, 2012; and U.S. Pat. Nos. 5,559,022, 5,672,346 and 5,827,735.

In addition to adding various osteoinductive or osteogenic agents, such as stem cells, to the bone implant compositions of the presently-disclosed subject matter, it is further contemplated that a number of additional therapeutic agents can also be added directly to the compositions. Further therapeutic agents that can be added to the bone implant compositions include, but are not limited to: collagen and insoluble collagen derivatives; hydroxyapatite; bisphosphonates and/or other anti-osteoporosis drugs; antivirals or viricides, such as those effective against HIV and hepatitis; amino acids, peptides, vitamins, and/or co-factors for protein synthesis; hormones; endocrine tissue or tissue fragments; synthesizers; enzymes, such as collagenase, peptidases, oxidases; polymer cell scaffolds with parenchymal cells; angiogenic drugs and polymeric carriers containing such drugs; collagen lattices; biocompatible surface active agents; antigenic agents; cytoskeletal agents; cartilage fragments; living cells, such as chondrocytes, bone marrow cells, mesenchymal stem cells; natural extracts; tissue transplants; bioadhesives; transforming growth factor (TGF-beta); insulin-like growth factor (IGF-1); parathyroid hormone; growth hormones, such as somatotropin; bone digesters; antitumor agents; fibronectin; cellular attractants and attachment agents; immuno-suppressants; and, permeation enhancers, e.g. fatty acid esters such as laureate, myristate and stearate monoesters of polyethylene glycol, enamine derivatives, and alpha-keto aldehydes.

In some embodiments, an antibiotic is added to the bone implant compositions of the presently-disclosed subject matter. Various antibiotics can be employed in this regard including, but are not limited to: aminoglycosides, such as amikacin, gentamycin, kanamycin, neomycin, netilmicin, paromomycin, streptomycin, or tobramycin; carbapenems, such as ertapenem, imipenem, meropenem; chloramphenicol; fluoroquinolones, such as ciprofloxacin, gatifloxacin, gemifloxacin, grepafloxacin, levofloxacin, lomefloxacin, moxifloxacin, norfloxacin, ofloxacin, sparfloxacin, or trovafloxacin; glycopeptides, such as vancomycin; lincosamides, such as clindamycin; macrolides/ketolides, such as azithromycin, clarithromycin, dirithromycin, erythromycin, or telithromycin; cephalosporins, such as cefadroxil, cefazolin, cephalexin, cephalothin, cephapirin, cephradine, cefaclor, cefamandole, cefonicid, cefotetan, cefoxitin, cefprozil, cefuroxime, loracarbef, cefdinir, cefditoren, cefixime, cefoperazone, cefotaxime, cefpodoxime, ceftazidime, ceftibuten, ceftizoxime, ceftriaxone, or cefepime; monobactams, such as aztreonam; nitroimidazoles, such as metronidazole; oxazolidinones, such as linezolid; penicillins, such as amoxicillin, amoxicillin/clavulanate, ampicillin, ampicillin/sulbactam, bacampicillin, carbenicillin, cloxacillin, dicloxacillin, methicillin, mezlocillin, nafcillin, oxacillin, penicillin G, penicillin V, piperacillin, piperacillin/tazobactam, ticarcillin, or ticarcillin/clavulanate; streptogramins, such as quinupristin/dalfopristin; sulfonamide/folate antagonists, such as sulfamethoxazole/trimethoprim; tetracyclines, such as demeclocycline, doxycycline, minocycline, or tetracycline; azole antifungals, such as clotrimazole, fluconazole, itraconazole, ketoconazole, miconazole, or voriconazole; polyene antifungals, such as amphotericin B or nystatin; echinocandin antifungals, such as caspofungin or micafungin, or other antifungals, such as ciclopirox, flucytosine, griseofulvin, or terbinafine. In some embodiments, the antibiotic that is included in a bone particle composition of the presently-disclosed subject matter is vancomycin. For further explanation and guidance regarding the use of cements, such as calcium phosphate cements, as drug delivery systems, see, e.g., Verron, et al. Drug Discovery Today. 15(13/14): 547-552 (2010).

Further provided, in some embodiments of the presently-disclosed subject matter, are kits including the components of the exemplary bone implant compositions. In some embodiments, a kit is provided that includes a biologically-resorbable cement powder, a viscous binder, and a plurality of bone microparticles, where each of the bone microparticles have a particle size of less than about 250 μm.

In some embodiments, the bone microparticles included in the kit are lyophilized or are otherwise dehydrated. In this regard, in some embodiments, the kit can further include a hydration liquid or vehicle for adding to the cement powder, the bone particles, or both the cement powder and bone particles. In some embodiments, and as indicated above, the biologically-resorbable cement powder and the plurality of bone microparticles are included in a first container, and the hydration liquid is included in a second container. In other embodiments, the biologically-resorbable cement powder is included in a first container, and the hydration liquid and the plurality of bone microparticles are included in a second container. In some embodiments, each of the components included in a respective kit can be metered before placing the components in their respective containers such that the entire contents of the containers can then be simply be combined for use. In some embodiments, the kit further comprises instructions for mixing the hydration liquid with the biologically-resorbable cement powder, the viscous binder, and/or the plurality of bone microparticles. In some embodiments, a product sub-kit is provided in an exemplary kit that further includes a mixing syringe or delivery system for mixing the product components in addition to any ancillary syringes and/or cannulas for delivery to the implant site.

In yet further embodiments of the kits described herein, one or more means for delivering the bone implant compositions are further included in the kits. In some embodiments, an exemplary kit further comprises a syringe for mixing or delivering the bone implant composition. In some embodiments, an exemplary kit further comprises a delivery cannula for delivery of the bone implant composition.

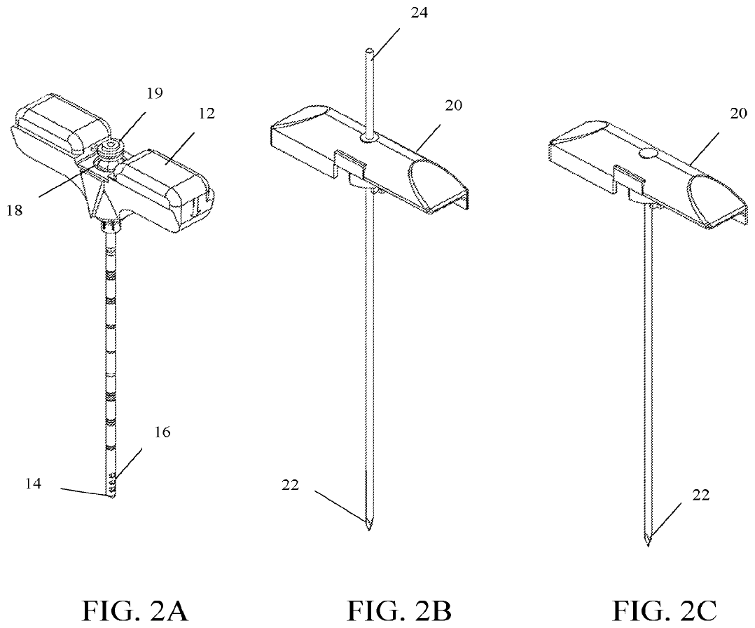

As one example of a delivery cannula included in an exemplary kit and/or is capable of use in accordance with the presently-disclosed subject matter, in some embodiments and as shown in FIGS. 2A-2E, a delivery cannula 10 is provided that is comprised of an outer cannula 12 with and open distal end 14 and with one or more holes 16 for injection of an exemplary bone implant composition. The delivery cannula 10 further includes a proximal end 18 with a Luer attachment 19. A second portion 20 of the delivery cannula then slides inside and attaches to the outer cannula 12 and has a sharp point at the distal end 22 so as to be able to pierce bone. The proximal end 24 of the second portion 20 is then capable of attaching to a drill or handle.

Still further provided, in some embodiments of the presently-disclosed subject matter, are methods for treating a bone defect. In some embodiments, a method for treating a bone defect is provided that comprises administering an effective amount of a bone implant composition of the presently-disclosed subject matter to a bone defect site in a subject.

As used herein, the terms “treatment” or “treating” relate to any treatment of a bone defect, including, but not limited to, prophylactic treatment and therapeutic treatment. As such, the terms “treatment” or “treating” include, but are not limited to: precluding a bone defect or the development of a bone defect; inhibiting the progression of a bone defect; arresting or precluding the development of a bone defect; reducing the severity of a bone defect; ameliorating or relieving symptoms associated with a bone defect; and causing a regression of the bone defect or one or more of the symptoms associated with the bone defect.

The term “bone defect” is used herein to refer to any bone tissue imperfection or discontinuity in the structure of a bone. For example, in some embodiments, the bone defect site is a bone void, or, in other words, an empty space that is typically occupied by bone. As another example, in some embodiments, the bone defect is a bone fracture or a disruption in the continuity of a bone. As yet another example, in some embodiments, the bone defect is within cancellous bone and can be a site of a localized pathology such as a bone marrow lesion, a bone edema, necrosis, a site of a cyst or tumor resection, and/or some other abnormality. In some embodiments, administering the bone implant composition can be further useful for augmenting the placement and/or fixation of bone hardware in the subject.

For administration of a bone implant composition disclosed herein, the bone implant compositions are typically administered in an amount sufficient to fill the site of the bone defect, i.e., an “effective amount.” Of course, the optimum amount of a bone implant composition used to fill a bone defect will vary depending on the size and/or shape of the particular bone defect being filled. However, determination and adjustment of the amount of a bone implant composition to be used in a particular application or subject, as well as when and how to make such adjustments, can be ascertained using only routine experimentation or clinical experience common to those skilled in the art.

In some embodiments, administering the bone implant composition comprises administering the bone implant composition through a syringe or cannula, such as the exemplary cannula outlined above. In some embodiments, administering the composition comprises applying a force of less than 100 N to a syringe plunger to inject the bone implant composition into a bone through a needle or cannula. Once administered, and upon the subsequent hardening or curing of the administered bone implant composition and without wishing to be bound by any particular theory or mechanism, it is believed that the hardened bone implant composition converts to hydroxyapatite or similar mineral phase, and the resulting bone implant has a compressive strength that is able to augment damaged cancellous bone, such as, in certain embodiments, a strength of greater than 0.1 MPa, greater than 1 MPa, greater than 2 MPa, greater than 3 MPa, greater than 4 MPa, greater than 5 MPa, greater than 6 MPa, greater than 7 MPa, greater than 8 MPa, greater than 9 MPa, greater than 10 MPa, or more. In this regard, it is thus believed that the exemplary bone implant compositions described herein can be effectively and efficiently used in the treatment of bone defects and can be utilized in a number of minimally invasive/percutaneous orthopedic procedures to treat such bone defects and associated pathologies in a subject whereby only a small incision or the insertion of an instrument such as a needle or cannula is required to deliver the bone implant composition to the site of the bone defect in the subject. In some embodiments, such delivery of the bone implant composition to the subject can be performed by first imaging the site of the bone defect (e.g., by magnetic resonance imaging (MRI)) prior to administering the bone implant composition.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The presently-disclosed subject matter is further illustrated by the following specific but non-limiting examples. Certain of the following examples are prophetic, notwithstanding the numerical values, results and/or data referred to and contained in the examples. The following examples may also include compilations of data that are representative of data gathered at various times during the course of development and experimentation related to the presently-disclosed subject matter.

### EXAMPLES

During the course and development of the presently-disclosed subject matter, and as described in further detail in the Examples below, very fine cortical allograft microparticles (less than 212 μm) were produced. These microparticles were designed to be similar to the particle sizes of a calcium phosphate cement (CPC) powder and were sufficiently small to fit into and flow through the porosity of cancellous bone. By properly combining the microparticles with the CPC and a viscous binder, it was observed that the mixture was injectable, remained cohesive and was capable of hardening at an implant site. In this regard, it was believed that not only would the presence of the allograft material provide osteoconductivity with osteoinductivity to promote bone formation and the healing of the lesion, but it was believed that the material would also provide a network of allograft bone throughout the synthetic graft and thereby increase the rate of the entire graft remodeling over time. The allograft material was expected to resorb and remodel first, while the remaining calcium phosphate continued to provide support and resorb more slowly. The allograft resorption was further believed to increase the surface area of the calcium phosphate exposed to osteoclasts and osteoblasts, allowing faster resorption and remodeling of the calcium phosphate as compared to synthetic only CPCs. This resorption rate was perceived as providing a significant and unexpected advantage as some products resorb too quickly, leaving behind unsupported bone/cartilage, while others resorb too slowly or not at all, preventing the lesion from ever healing.

### Example 1—Production of Bone Microparticles and Bone Implant Compositions

Precise demineralization of the small bone microparticles posed several challenges as compared to larger particles. Milling the bone into microparticles post-demineralization dramatically reduced osteoinductivity, as the mineral phase of the bone tissue served to protect collagen from high temperatures and (collagen) protein denaturation during the milling processes, and thus was not a viable option. Previous demineralized bone particle technology used larger particles of bone, so separation of the solid particles from the liquid chemicals in the cleaning and demineralization processes has to date typically been quick and simple, and done by pouring the mixture through a sieve or filter. However, microparticles are generally so fine, that when mixed with a liquid as part of cleaning and demineralizing processes, the microparticles stay in suspension in the liquids. Any attempts to filter the material, even under vacuum, has previously resulted in the microparticles clogging the filter and restricting the liquid from passing through. It was further observed that it was difficult to remove the fine particles from any type of filter without significant reduction in yield. To compound this issue, during the demineralization step, where the calcium was removed from the tissue exposing the collagen, the particles often change consistency and become softer, stickier, and tended to adhere together, further complicating the separation/filtering process. Additionally, for the same mass of bone, the particle size of the bone microparticles resulted in a surface area much greater than the surface area of larger particles, requiring significantly less exposure time. So, it was observed that the inability to quickly separate the bone microparticles from the liquid made controlling the timing of acid exposure to the tissue difficult, leading to unpredictable levels of demineralization and/or overexposure and denaturation of the proteins and collagen. The inability to sieve or filter the material also made ‘rinsing’ the particles with water or other solutions post-demineralization difficult, which again generally resulted in overexposure and excess demineralization. Due to all these challenges, efforts to scale up and retain sufficient yield of the microparticles through the entire process has, to date, been difficult.

To address these and other issues, experiments were undertaken to analyze the ability to use bone that was milled into microparticles prior to demineralization. Moreover, in these experiments and during cleaning and demineralization, a centrifuge was used to force the particles out of liquid suspension and compact the particles into a plug of material at the bottom of a centrifuge container. This allowed any liquid to then be decanted/poured off in a timely manner without losing significant yield or causing overexposure. Additionally, a specific acid concentration (e.g., 1N) was used to maximize the processing capacity without causing damage to the tissue's mechanical and biological properties. Rather than relying on timing to control the demineralization reaction, the quantity (i.e., the number of moles) of acid was also used to control the amount of calcium that was removed.

Briefly, to produce a bone implant composition in accordance with the presently-disclosed subject matter, human cortical bone shafts from a donor's femur, tibia, fibula, humerus, radius, and/or ulna were obtained and were then debrided and cleaned using a series of lavages and soakings in detergents, antibiotic washes, hydrogen peroxide, isopropyl alcohol and sterile water. The cortical shafts were then lyophilized to remove residual moisture and broken into smaller pieces (approximately 2 mm-50 mm), using, for example, a hammer and chisel, a piston-cylinder crushing tool, cutting mills such as the Fritsch Pulverisette 19 (Fritsch Milling and Sizing Inc., Pittsboro, NC), or jaw crusher mill such as the Fritsch Pulverisette 1 (Fritsch Milling and Sizing Inc., Pittsboro, NC).

Using the obtained and produced smaller bone pieces, approximately 35 grams of bone was then placed in a centrifugal mill (Fritsch Pulverisette 14, Fritsch Milling and Sizing Inc., Pittsboro, NC, 24-bit rotor, 0.08 mm sieve ring) with a cyclone attachment, and was milled at 20,000 RPM. The bone microparticles were then removed from the collection container and sieved to capture bone particles having a particle size less than 212 μm with an approximate 97% wt. yield, as it was later experimentally determined that without the sieving step the approximately 3% of bone microparticles having particle sizes greater than 212 μm resulted in a composition that failed to provide a suitable injection profile. Particle size analysis was conducted on the bone microparticles produced using this milling procedure via x-ray diffraction with liquid dispersion, and the bone microparticles exhibited a mean particle size of 26.0 μm and a distribution of D(10)=3.49 μm, D(50)=13.4 μm, and D(90)=65.3 μm.

In another milling procedure, approximately 35 grams of the initially obtained small bone pieces were placed in a blade grinder consisting of a modified YF2-1 Automatic Medicinal Herbs Grinder (iPharMachine, Garden Grove, CA). The bone was then milled using 8×30 second bursts, as it was observed that some lesser amounts or shorter bursting times resulted in larger particles not capable of being adequately injected as described below. Approximately 1 hour of wait time was observed in between each burst, for a total of 4 minutes of milling, as it was also observed that in certain procedures, longer milling intervals (e.g., greater than 30 seconds) generated excess heat about 40° C. that subsequently resulted in decreased osteoconductivity of the particles. The bone microparticles were then removed from the grinder and sieved to capture bone particles having a particle size less than 212 μm with an approximate 98.0% wt. yield. Particle size analysis was conducted on the bone microparticles produced using this alternative milling procedure via x-ray diffraction with liquid dispersion, and the resulting bone microparticles exhibited a mean particle size of 36.2 μm and a distribution of D(10)=3.65 μm, D(50)=19.2 μm, and D(90)=94.7 μm.

In a further milling procedure, approximately 35 grams of the initially obtained small bone pieces were placed in a hammer mill consisting of a YF3-1 Automatic Continuous Herbs Grinder with 150 mesh (iPharMachine, Garden Grove, CA). The bone was then milled for 1 minute 45 seconds. The bone microparticles were subsequently removed from the collection vessel and sieved to capture bone particles having a particle size less than 212 μm with an approximate 98.0% wt. yield. Particle size analysis was again conducted on the bone microparticles produced using this further milling procedure via x-ray diffraction with liquid dispersion, and the resulting bone microparticles exhibited a mean particle size of 76.4 μm and distribution of D(10)=8.01 μm, D(50)=54.9 μm, and D(90)=178 μm. However, it was later observed that the bone microparticles produced using this later milling procedure resulted in a particle sizes and/or distribution that was not capable of achieving certain injection profiles, as also described below.

Upon the production of the bone microparticles by the milling procedures described above, the bone microparticles underwent a demineralization process whereby 30 grams of bone microparticles obtained from a particular milling procedure were placed into a 750 mL centrifuge bottle. 120 mL of 70% ethanol was then first added, and the mixture was agitated on a vortex mixer and mixed using an orbital shaker at 200 RPM for 10 minutes. The sample was then centrifuged at 4000 RPM for 11 minutes and the excess ethanol was decanted and poured off. 310 mL of 1 N hydrochloric acid was next added, the mixture was agitated on a vortex mixer and then mixed using an orbital shaker at 200 RPM for 10 minutes. The sample was then again centrifuged at 4000 RPM for 11 minutes and the excess acid was decanted and poured off.

Following the addition and removal of the acid, a neutralization step was used where a total of 630 mL of 0.1 M potassium phosphate buffer (KPB) was slowly added over 10 minutes while agitating. This step brought the pH of the solution to a pH of approximately 7.0, which improved biocompatibility and osteoinductivity. The sample was then centrifuged once more at 4000 RPM for 11 minutes and the excess buffer was decanted and poured off. 310 mL of USP water was added as a washing step, and the mixture was agitated on a vortex mixer and then mixed using an orbital shaker at 200 RPM for 10 minutes. The sample was then further centrifuged at 4000 RPM for 11 minutes, and the excess water was decanted and poured off. This was repeated two more times, for a total of 3 water dilutions.

The demineralized bone particles produced using the above-described procedure subsequently underwent a post-processing, elemental analysis using Inductively Coupled Plasma Optical Emission spectroscopy (ICP-OES), and that analysis demonstrated a residual calcium content of 4.39% using the above described procedures. Similar procedures were also used to produce partially demineralized bone microparticles having a residual calcium content of 12.7% by making use of 242 mL HCl and 530 mL of KPB, or having a residual calcium content of 19.9% by making use of 162 mL HCl and 400 mL of KPB. Non-demineralized bone microparticles were also produced by not subjecting the bone microparticles to the above-described demineralization procedures.

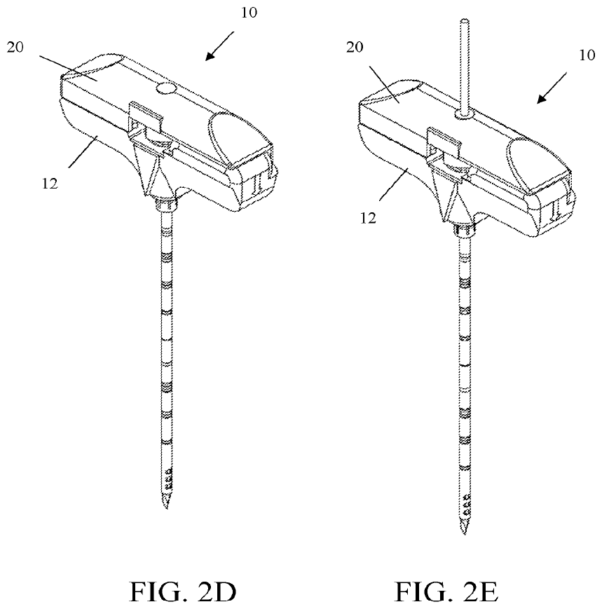

During the development of the demineralization procedures, studies were also completed that demonstrated that for specific particle sizes, acid concentrations and neutralization agents, a linear relationship between the amount of acid added and the calcium content could be defined. In particular, during the demineralization process and as shown in FIG. 3, pH was measured to monitor the reaction's progress. Using a prescribed quantity of acid to control the level of demineralization, rather than timed exposure to acid, distinct different levels of demineralization could be achieved within the same time frame, while avoiding overexposure to the acid and subsequent denaturation of the bone microparticles. For the particle sizes described herein, most of the reaction occurred within two minutes of exposure to 1N acid. By five minutes, the pH started to plateau but not all the calcium had been removed. By 10 minutes, the pH stabilized and the reaction was completed.

Also during the demineralization process, specified quantities and concentrations of acid were reacted with the bone microparticles to identify the amounts needed to remove the desired amount of calcium without having an adverse effect on the particles. As shown in FIG. 4, a linear relationship was observed between the acid to bone ratio and the residual calcium content for a 10 minute exposure to 1M acid for 30 grams of bone in the particle size distributions described herein.

To preserve the demineralized bone microparticles until use, 55 mL of USP water was added to the post-demineralized sample, agitated on a vortex mixer and transferred into a container compatible with lyophilization. This created a slurry of material which could be poured into collection vessels, rather than physically transferring the wet plug material, which results in yield loss. The samples were then subjected to lyophilization to remove the liquid, resulting in a dry demineralized bone microparticle powder that can be incorporated into the calcium phosphate powder.

After producing the bone microparticles having varying levels of demineralization, injectable bone implant compositions of the presently-disclosed subject matter were then produced by combining the bone microparticles with varying amounts of calcium phosphate powder, a sodium phosphate dibasic solution, and an amount of carboxymethyl cellulose (CMC) powder.

As one example, an injectable bone implant composition comprised of 20% demineralized bone microparticles and having a calcium content of 4.39% was produced by combining 1.200 grams of calcium phosphate powder, 0.300 grams of demineralized bone microparticles, 1.575 mL of sodium phosphate dibasic (0.175M) solution, and 0.070 grams of CMC powder.

As another example, an injectable bone implant composition comprised of 20% partially demineralized bone microparticles and having a calcium content of 12.7% was produced by combining 1.500 grams of calcium phosphate powder, 0.375 grams of demineralized bone microparticles, 1.481 mL of sodium phosphate dibasic (0.175M) solution, and 0.059 grams of CMC powder.

As another example, an injectable bone implant composition comprised of 20% partially demineralized bone microparticles and having a calcium content of 19.9% Ca was produced by combining 1.200 grams of calcium phosphate powder, 0.300 grams of demineralized bone microparticles, 1.050 mL of sodium phosphate dibasic (0.175M) solution, and 0.037 grams of CMC powder.

As another example, an injectable bone implant composition comprised of 50% demineralized bone microparticles and having a calcium content of 4.39% was produced by combining 0.600 grams of calcium phosphate powder, 0.600 grams of demineralized bone microparticles, 1.848 mL of sodium phosphate dibasic (0.175M) solution, and 0.081 grams of CMC powder.

As another example, an injectable bone implant composition comprised of 50% partially demineralized bone microparticles and having a calcium content of 12.7% was produced by combining 0.675 grams of calcium phosphate powder, 0.675 grams of demineralized bone microparticles, 1.526 mL of sodium phosphate dibasic (0.175M) solution, and 0.061 grams of CMC powder.

As another example, an injectable bone implant composition comprised of 50% partially demineralized bone microparticles and having a calcium content of 19.9% was produced by combining 0.900 grams of calcium phosphate powder, 0.900 grams of demineralized bone microparticles, 1.782 mL of sodium phosphate dibasic (0.175M) solution, and 0.091 grams of CMC powder.

As another example, an injectable bone implant composition comprised of 50% non-demineralized bone microparticles and having a calcium content of 25% was produced by combining 1.200 grams of calcium phosphate powder, 1.200 grams of non-demineralized bone microparticles, 1.608 mL of sodium phosphate dibasic (0.175M) solution, and 0.099 grams of CMC powder.

### Example 2—Analysis of Injectability of Bone Implant Composition

Many biomaterials are claimed to be “injectable” but only demonstrate flow through a specific gauge/diameter cannula or an open cancellous bone-like network. However in vivo, cancellous bone is not a typical open system, rather a network of plates and bar-like structures filled with bone marrow and framed by dense cortical bone on at least one side. Thus, and without wishing to be bound by any particular theory or mechanism, it was believed that to successfully achieve injection of a material into a cancellous bone region, the material had to be able to be pressurized and flow hydraulically through the trabecular network, pushing/displacing the bone marrow and any blood, edema or lipids out of the trabecular gaps and pores. Additionally, it was believed that the particle size had to be small enough to fit through the trabecular gaps, with a particle size distribution and packing fraction sufficient to enable the flow of the particles through the microarchitecture.

In order to assess whether the bone implant compositions were capable of achieving such injectability, appropriate proportions, including those exemplified above, of the bone microparticles, calcium phosphate powder, CMC, and liquid are weighed and combined. All components were mixed together for one minute to create a paste and where then transferred into a 1 mL syringe(s).

Two benchtop tests were then performed to assess the injectability. In the first test, the material was expelled from the 1 mL syringe(s) through a 1.5 inch long, 21-gauge needle into open air. The needle, having an internal diameter of around 500 μm, was representative of trabecular separation within dense cancellous bone and was a clinically relevant challenge of the pore size the material must be able to flow through. For additional information and histomorphometric measurements of trabecular bone structures, see, e.g., Klintstrom, et al. Dentomaxillofac Radiol. December 2014; 43(8): 20140196, which is incorporated herein by reference in its entirety.

In the second benchtop test, a rigid cellular foam block designed for biomechanical testing (Pacific Research Laboratories cellular foam: [1522-11]12.5 pcf or [1522-1300]15 pcf, Pacific Research Laboratories, Vashon, WA) was used. The foam was cut into 4 cm cubes, and an 11-gauge delivery cannula with fenestrations was placed into the block and the exemplary bone implant compositions were pushed from the 1 mL syringe(s), through the cannula and into the foam. To achieve this, the biomaterial had to be able to push and break through the cell walls of the foam, interdigitating throughout the block.

If that material formulation exhibited sufficient injectability, in both tests, 100% of the material would be able to be expelled from the syringe using only manual/digital pressure with less than about 100N of force on the syringe plunger, and without leaving any un-extrudable material behind in the syringe. If the material formulation was insufficient, the bone implant compositions would either clog the needle or cannula, phase separate and become un-extrudable, or would require too much force on the syringe plunger to facilitate injection by hand.

Upon analysis of the results from the experiments, it was observed that the bone implant compositions including the bone microparticles disclosed herein, were able to pass both injection tests. Specifically, all formulations with component ratios described in Example 1 made with appropriate size particles, such as those produced by milling using the blade grinder procedure outline in para. [0089] above, passed both injection tests. It was further observed, however, that formulations with too much viscous binder failed injection due to too high of injection pressure, whereas formulations with too little viscous binder fails injection due to phase separation. Formulations with too little hydration liquid resulted in dry and un-extrudable paste, whereas formulations with too much hydration liquid resulted in a runny paste prone to phase separation with prolonged setting. Without wishing to be bound by any particular theory or mechanism, it was believed that microparticles with higher calcium content were more ridged and more prone to injection failures, whereas microparticles with lower calcium content were less ridged and easier to pass injection testing.

Additionally, these injectability testing methods were confirmed to correlate to successful in situ injection into human cadaver cancellous bone, including the distal femur, proximal tibia, femoral head, talus and calcaneus.

### Example 3—Setting Characteristics of Bone Implant Compositions

To analyze the setting characteristics of the exemplary bone implant compositions described herein, various bone implant compositions were prepared according to the examples disclosed herein and were extruded through a 21-gauge needle into a simulated open void bone defect. The top surface of the cement was smoothed flat and, at 10 minutes, the filled defect was submerged into 36-37° C. phosphate buffered saline bath to simulate an in vivo environment to allow for setting. Starting at 13 minutes, a Gillmore Needle Apparatus (Gilson Company, Inc. Lewis Center, OH), which consisted of a 0.25 pound, 2.12 mm diameter needle apparatus, was lowered onto the surface of each tested implant composition. The indentation of the needle was observed and, if the indentation was a complete circle, the test was repeated every minute until there was an incomplete circular indentation, or until no indentation was left. The time at which the indentation was less than a complete circle was recorded as Initial Set Time.

After the composition passed the Initial Set Time, a Gillmore Needle Apparatus consisting of a 1.0 pound, 1.06 mm diameter needle was lowered onto the composition surface. The indentation of the needle was observed and, if the indentation was a complete circle, the test was repeated every minute or, as determined applicable by the operator, until there was an incomplete circular indentation or until no indentation was left. The time at which the indentation was less than a complete circle was recorded as Final Set Time. The testing for both the Initial Set Time and the Final Set Time was performed in accordance with ASTM C266, Standard Test Method for Time of Setting of Hydraulic-Cement Paste by Gillmore Needles.

Upon analysis of the results from these experiments, it was observed that for the initial set tests, exemplary bone implant compositions including up to 50% bone microparticles passed Initial Set in under 1 hour, with the exemplary bone implant composition including up to 25% bone microparticles passing the Initial Set in under 30 minutes. For the Final Set Time, exemplary bone implant compositions including up to 50% demineralized bone microparticles did not pass the Final Set, while exemplary bone implant compositions including up to 50% non-demineralized and 25% demineralized or non-demineralized bone microparticles passed the Final Set in under 12 hours.

The analysis of the setting characteristics of these experiments revealed that compositions with increased amounts of bone microparticles by weight had increased set times. Without wishing to be bound by any particular theory or mechanism, this was believed to be because the presence of additional aggregate material within the calcium phosphate mixture affected the crystallization process. In general, compositions including non-demineralized bone microparticles had a shorter set time than compositions including demineralized bone microparticles, and the more demineralized the particles (i.e. the lower the calcium content), the longer the set times. By removing the calcium mineral from the bone in the demineralization process, such a removal decreased the mechanical strength of the tissue and required more liquid to hydrate the tissue, which was thought to prolong and affect the crystallization of the calcium phosphate and cause the biomaterial to be unable to withstand as much load.

### Example 4—Analysis of Dimensional Stability of Bone Implant Compositions

To assess the dimensional stability of the bone implant compositions, the exemplary bone implant compositions outlined in Example 1 were prepared and expelled from the syringe through a needle and into cylindrical molds approximately 6 mm in diameter and 12 mm in height. The material was allowed to set and after 2 hours the samples were removed from the molds and the initial length of the cylinder was measured. The cylinders were then placed into a simulated in vivo environment (PBS bath at 37° C.) after which the final length of material was measured after 24 hours. Dimensional stability was calculated as the percentage difference in length over the 24-hour period.

In reviewing the results obtained from the various bone implant compositions, it was observed that the exemplary bone implant compositions including 25% bone microparticles demonstrated less than a 1.5% change in length, whereas compositions including 50% bone microparticles demonstrated less than a 2.5% change in length. In both instances, the exemplary bone implant compositions including non-demineralized bone microparticles demonstrated less change in length than the compositions including demineralized bone microparticles. All compositions remained intact, however, and maintained their initial shape in the simulated in vivo environment without significant swelling or distortion. Each of the exemplary compositions were considered dimensionally stable, and thus did not present concern for migration during in vivo use.

### Example 5—Compressive Strength of Exemplary Bone Implant Compositions

To test the compressive strength of the compositions, each of the exemplary bone implant compositions described in Example 1 were prepared and were subsequently expelled from a syringe through a needle and into cylindrical molds approximately 6 mm in diameter and 12 mm in height. The compositions were then removed from the molds and allowed to set. One group for each composition was allowed to set submerged in a PBS bath at 37° C. for approximately three days, whereas another group for each composition was allowed to set in open air for approximately three days prior to compression testing. A mechanical test fixture, consisting of two opposing rigid platens, loaded the cylinders in compression axially while under displacement control at a rate of 0.5 mm/min until failure. Compressive strength (ultimate stress) was then recorded as the maximum amount of load reached during the test.

TABLE 1
Compressive Strength for Selected Implant Compositions

| | | | Compressive | Compressive |
|---|---|---|---|---|
| Amount | | Amount | Strength - Wet | Strength - Dry |
| Bone* | Level of Demineralization | CaP* | (MPa ± SD) | (MPa ± SD) |
| 25% | Demineralized (4.39% Ca) | 75% | 0.98 ± 0.04 | 4.36 ± 0.17 |
| 25% | Partially Demineralized (12.7% Ca) | 75% | 1.36 ± 0.08 | 8.29 ± 0.45 |
| 25% | Partially Demineralized (19.9% Ca) | 75% | 1.94 ± 0.12 | 10.89 ± 0.53 |
| 50% | Demineralized (4.39% Ca) | 50% | 0.23 ± 0.01 | 2.04 ± 0.11 |
| 50% | Partially Demineralized (12.7% Ca) | 50% | 0.21 ± 0.02 | 3.29 ± 0.57 |
| 50% | Partially Demineralized (19.9% Ca) | 50% | 0.32 ± 0.03 | 4.09 ± 0.21 |
| 50% | Non-Demineralized (25% Ca) | 50% | 1.37 ± 0.14 | 8.82 ± 1.30 |

*Amount of Bone Microparticles and Calcium Phosphate (CaP) powder are provided in weight percentage of dry powder components.

Upon analysis of the results, it was observed that increasing the amount of bone in the compositions decreased the compressive strength, and generally the strength of the implant also decreased with the lower calcium content of the bone microparticles. However, this difference was less pronounced in the wet environment such that the compressive strength in vivo was believed to be somewhere in between these two conditions. All the exemplary implant compositions tested exhibited potential to augment cancellous bone defects.

### Example 6—In Vitro Osteoinductivity of Bone Implant Compositions

To analyze osteoinductivity, the osteoinductivity of the bone morphogenetic proteins (BMPs) present in the exemplary bone implant compositions described in Example 1 were measured by assessing alkaline phosphatase (ALP) activity during the co-incubation of the samples with a C2C12 cell line (see, e.g., Han, et al, Orthop Res. 2003 July; 21(4):648-54, which is also incorporated herein by reference in its entirety), as correlation studies have been performed and demonstrate the ALP activity induced by the C2C12 cell line correlates to new bone formation in vivo and have suggested that an in vitro score greater than or equal to 0.2 can be considered osteoinductive in an in vivo model.

Exhaustive methods of preparing the bone microparticles, including various milling, cleaning and demineralization procedures, were tested for their effects on osteoinductivity in vitro. For each of these studies, samples of the bone microparticles (with varying levels of demineralization) and pre-set 0.2 cc cylinders of the bone implant compositions were used. It was generally observed that the milling method and temperature had a profound effect on osteoinductivity, especially when milling to particles sizes less than 106 μm. Milling at temperatures greater than 40° C. pre- or post-demineralization also was observed to reduce and/or eliminate osteoinductivity. Low temperature milling post-demineralization also reduced and eliminated osteoinductivity. Smaller particle sizes had less osteoinductivity as compared to larger particles sizes, yet bone particles of 0.1 μm to 212 μm were still observed to be osteoinductive when processed according to the methods described herein. Further, exposure of bone microparticles to peroxide reduces osteoinductivity

In the experiments, the timing required to demineralize the microparticles was of course proportional to particle size; however, the amount of acid required to demineralize a prescribed mass of bone to a specified level of demineralization increased with particle size (i.e. the acid to bone ratio for a given calcium content changes dependent on particle size). It was further observed that overexposure to acid (e.g., approximately 20 minutes or more) decreased osteoinductivity. Decreasing the HCl concentration (e.g., less than 1 N), did not improve the osteoinductivity of the microparticles, but concentrations over 1 N affected both the mechanical and biological properties. Neutralizing the bone microparticles to a pH of approximately 7 improved osteoinductivity, and it was further observed that the type, quantity, and concentration of neutralization agents affected osteoinductivity. It was also observed that neutralization with potassium phosphate buffer improves osteoinductivity compared to phosphate buffered saline, sodium bicarbonate and water.

In the osteoinductivity tests, utilizing the methods and compositions described herein, demineralized bone microparticles of varying calcium content (including about 0, 2, 5, 12, and 20% Ca) demonstrated positive osteoinductivity scores. Furthermore, in some instances, the compositions of the demineralized bone microparticles in the calcium phosphate cement demonstrated enhanced osteoinductivity over the bone microparticles alone, even when the calcium phosphate alone was non-osteoinductive. For example, the calcium phosphate alone scored a 0.078, and a sample of demineralized microparticles (2.67% calcium) alone scored 0.586, whereas when the same microparticles incorporated in the calcium phosphate cement scored a 3.065. Interestingly, the level of demineralization (calcium content) did not affect osteoinductivity as much as did the percent weight of bone microparticles included in the composition. For example, compositions including 50% wt. of demineralized microparticles of 4.39% calcium scored a 2.121, and a composition including 50% wt. demineralized microparticles of 19.9% calcium scored a 1.579. Whereas composition of the same bone microparticles at 25% wt. composition scored 0.070 and 0.087, respectively. This indicated that even partially demineralized bone microparticles at sufficient weight composition were a viable option for a growth promoting implant. In some compositions, the incorporation of bone into the calcium phosphate decreased the osteoinductivity from the bone alone, and the compositions including non-demineralized bone were considered non-osteoinductive. However, even compositions that did not test positive for osteoinductivity were not excluded from consideration, since the inclusion of bone microparticles in a calcium phosphate cement was expected to likely remodel into bone faster that a calcium phosphate or synthetic composition alone, and would still be considered superior for bioactivity as compared to any commercially-available injectable self-setting cement.

### Example 7—In Vivo Analysis of Bone Implant Compositions

In vivo analysis of the exemplary bone implant compositions is performed using an industry accepted method for assessing osteoinductivity of a demineralized bone matrix, namely an ectopic bone formation model originally described by Marshal Urist and now outlined by ASTM F2529. This method determines whether a material is able to cause bone to form in vivo at a site that would otherwise not support bone formation. For this study, the implant materials are inserted into pouches made in the hind limbs or abdominal muscles of athymic rats. 28 days after implantation, the animals are euthanized and the implant location is excised and decalcified and processed for histology. Implant sites are scored for new bone formation as follows: 0=no implant detected upon explant, fails test; 1=<10% new bone forming elements, fails test; 2=10-20% new bone forming elements, passes test; 2=21-30% new bone forming elements, passes test; 2=>30% new bone forming elements, passes test.

For this study, both the bone microparticles themselves (of varying levels of demineralization) and pre-set 0.2 cc cylinders of the bone implant compositions (varying in composition) are utilized and, upon the analysis of the results from this experiments, it is observed that the in vivo results correlate with the data obtained with the in vitro osteoconductivity experiments described above.

All publications, patents, and patent applications mentioned in this specification are herein incorporated by reference to the same extent as if each individual publication, patent, or patent application was specifically and individually indicated to be incorporated by reference, including the reference(s) set forth in the following list:

### REFERENCES

U.S. Pat. No. 10,046,090, to Vivorté, Inc., issued Aug. 14, 2018, and entitled “Processed Bone Particle Compositions and Related Methods.”

It will be understood that various details of the presently disclosed subject matter can be changed without departing from the scope of the subject matter disclosed herein. Furthermore, the foregoing description is for the purpose of illustration only, and not for the purpose of limitation.

1.-65. (canceled)

66. A bone implant composition, comprising:
a plurality of bone microparticles, each of the bone microparticles having a particle size of less than about 250 μm; and
a viscous binder.

67. The bone implant composition of claim 66, wherein the bone microparticles comprise allograft bone microparticles.

68. The bone implant composition of claim 66, wherein the bone microparticles are at least partially demineralized.

69. The bone implant composition of claim 66, wherein each of the bone microparticles have a particle size of less than about 212 μm.

70. The bone implant composition of claim 66, wherein the plurality of bone microparticles has a mean particle size of less than about 35 μm.

71. The bone implant composition of claim 66, wherein the plurality of bone microparticles has a particle size distribution such that about ninety percent of the bone microparticles have a particle size of about 100 μm or less, about 75 μm or less, or about 50 μm or less.

72. The bone implant composition of claim 66, wherein the plurality of bone microparticles has a particle size distribution such that about fifty percent of the bone microparticles have a particle size of about 70 μm or less, about 30 μm or less, about 20 μm or less, or about 5 μm or less.

73. The bone implant composition of claim 66, wherein the plurality of bone microparticles has a particle size distribution such that about ten percent of the bone microparticles have a particle size of about 10 μm or less.

74. The bone implant composition of claim 66, wherein the viscous binder comprises cellulose.

75. The bone implant composition of claim 74, wherein the viscous binder comprises sodium carboxymethyl cellulose.

76. The bone implant composition of claim 66, further comprising a biologically-resorbable cement.

77. The bone implant composition of claim 76, wherein the biologically-resorbable cement is a calcium-based cement.

78. The bone implant composition of claim 77, wherein the calcium-based cement is a calcium phosphate cement or a calcium sulfate cement.

79. The bone implant composition of claim 78, wherein the calcium-based cement is a calcium phosphate cement, and wherein the calcium phosphate cement has a calcium:phosphate ratio of about 1.67 in a powder phase.

80. The bone implant composition of claim 76, wherein the biologically-resorbable cement is in the form of a powder and wherein the powder comprises about 40 wt. % to about 90 wt. % of a combined amount of the biologically-resorbable cement and the bone microparticles.

81. The bone implant composition of claim 76, wherein the bone microparticles comprise about 10 wt. % to about 60 wt. % of a combined amount of the biologically-resorbable cement and the bone microparticles.

82. The bone implant composition of claim 76, wherein the biologically-resorbable cement comprises resorbable ceramic powder.

83. The bone implant composition of claim 66, further comprising a synthetic bone graft extender.

84. The bone implant composition of claim 66, further comprising a hydration liquid.

85. The bone implant composition of claim 84, wherein the viscous binder is included in an amount comprising about 0.1 wt. % to about 15 wt. % of the hydration liquid.

86. The bone implant composition of claim 84, wherein the hydration liquid comprises a phosphate solution, a biological fluid, a saline solution, or water.

87. The bone implant composition of claim 86, wherein the phosphate solution is a sodium phosphate dibasic solution.

88. The bone implant composition of claim 86, wherein the biological fluid is selected from the group consisting of blood, plasma, blood serum, platelet rich plasma, bone marrow aspirate (BMA), or bone marrow aspirate concentration (BMAC).

89. The bone implant composition of claim 84, further comprising a biologically-resorbable cement, wherein the hydration liquid comprises about 35 wt. % to about 170 wt. % of a combined amount of the biologically-resorbable cement and bone microparticles included in the composition.

90. A bone implant composition, comprising:
a plurality of bone microparticles, each of the bone microparticles having a particle size of less than about 250 μm;
a viscous binder; and
a biologically-resorbable cement powder; and
a hydration liquid.

91. A kit including a bone implant composition, the kit comprising:
a biologically-resorbable cement powder;
a viscous binder; and
a plurality of bone microparticles, each of the bone microparticles having a particle size of less than about 250 μm.

92. The kit of claim 91, further comprising a hydration liquid.

93. The kit of claim 91, further comprising instructions for mixing the hydration liquid with the biologically-resorbable cement powder, the viscous binder, and/or the plurality of bone microparticles.

94. The kit of claim 91, further comprising a delivery cannula for delivery of the bone implant composition.

95. The kit of claim 91, further comprising a syringe for mixing or delivering the bone implant composition.

96. A method of treating a bone defect, comprising administering an effective amount of a bone implant composition of claim 1 to a site of a bone defect in a subject.

97. The method of claim 96, wherein the defect is a bone void, a fracture, osteonecrosis, bone edema, a bone marrow lesion, a cyst or a lytic lesion.

98. The method of claim 96, wherein administering the bone implant composition comprises administering the bone implant composition through a syringe or cannula.

99. The method of claim 98, wherein administering the composition comprises applying a force of less than 100 N to the syringe or cannula to inject the bone implant composition into a bone.

100. A method of making a bone implant composition, comprising:
obtaining an amount of intact bone;
milling the intact bone to produce a plurality of bone microparticles; and
demineralizing the plurality of bone microparticles by exposing the plurality of bone microparticles to an amount of acid and neutralizing the acid, the plurality of bone microparticles undergoing a step of centrifuging the plurality of bone microparticles both after exposing the bone microparticles to an amount of acid and after neutralizing the acid.

101. The method of claim 100, further comprising a step of rinsing the plurality of bone microparticles prior to exposing the bone microparticles to an amount of acid and subsequent to neutralizing the acid.

102. The method of claim 100, further comprising a step of lyophilizing the plurality of bone microparticles subsequent to demineralization.

103. The method of claim 100, wherein exposing the bone microparticles to an amount of acid comprises exposing the bone microparticles to an amount of acid calculated to remove a predetermined amount of calcium from the plurality of bone microparticles.
